(19) United States
(12) Patent Application Publication
(10) Pub. No.: US 2017/0319649 A1
(43) Pub. Date: Nov. 9, 2017

(54) NOVEL PEPTIDE ACTIVATOR OF CYCLIN C-DEPENDENT KINASE 8 (CDK8)

(71) Applicants: Thomas G. BOYER, San Antonio, TX (US); Jason M. SPAETH, San Antonio, TX (US); Alison D. CLARK, San Antonio, TX (US)

(72) Inventors: Thomas G. BOYER, San Antonio, TX (US); Jason M. SPAETH, San Antonio, TX (US); Alison D. CLARK, San Antonio, TX (US)

(73) Assignee: The Board of Regents of the University of Texsas System, Austin, TX (US)

(21) Appl. No.: 15/526,643

(22) PCT Filed: Nov. 19, 2015
(86) PCT No.: PCT/US2015/061522

§ 371 Date: May 12, 2017

Related U.S. Application Data
(60) Provisional application No. 62/081,983, filed on Nov. 19, 2014.

(51) Int. Cl. A61K 38/16

(52) U.S. Cl. CPC A61K 38/16

(57) ABSTRACT

Certain embodiments are directed to compositions and methods for treating conditions associated Med12 mutations. Certain embodiments are directed to a peptide comprising all or part of an amino acid sequence that is at least 90% identical to the ammo acid sequence of MAAFGILSYEHRPLKRPRLGPPDVYPQDPKQKEDELTALNVKQGFNNQPAVSGDEHGSAKNVSFNPAKISSNFSSIIAEKLRCNTLPDT (SEQ ID NO:1). In certain aspects a peptide can comprise 10, 20, 30, 40, 50, 60, 70, 80, 90 or 100 consecutive amino acids that is 90, 95, or 100% identical SEQ ID NO:1. In certain embodiments a peptide described herein can be comprised in a pharmaceutical composition. Certain aspects are directed to an expression vector encoding a peptide as described herein.

This application claims the benefit of priority to U.S. Provisional Patent Application Ser. No. 62/081,983, filed Nov. 19, 2014, which is hereby incorporated by reference in its entirety.

### STATEMENT REGARDING FEDERALLY FUNDED RESEARCH

This invention was made with government support under R01 MH085320 awarded by the NIH/NIMH. The government has certain rights in the invention.

### REFERENCE TO SEQUENCE LISTING

A sequence listing required by 37 CFR 1.821-1.825 is being submitted electronically with this application. The sequence listing is incorporated herein by reference.

### BACKGROUND

Uterine leiomyomas (fibroids) are monoclonal neoplasms of the myometrium and represent the most common pelvic tumor in reproductive age women (Stewart, 2001, Lancet, 357, 293-298). Although benign, they are nonetheless associated with significant morbidity. Uterine leiomyomas are the primary indicator for hysterectomy, and a major cause of gynecologic and reproductive dysfunction, ranging from profuse menstrual bleeding and pelvic discomfort to infertility, recurrent miscarriage, and pre-term labor (Stewart, 2001, Lancet, 357, 293-298).

Mutations in exons 1 and 2 of the Xq13 gene encoding the transcriptional Mediator subunit MED12 occur at very high frequency (˜70%) in uterine leiomyomas (Makinen et al., 2011, Science, 334, 252-255; Kämpjärvi et al., 2014, Hum Mutat, 35, 1136-1141). Along with their high-frequency occurrence, two additional genetic findings suggest that MED12 mutations likely contribute to the genesis of uterine leiomyomas. First, all observed MED12 exon 1 and 2 mutations affect highly evolutionarily conserved regions of the MED12 protein, including three principal hotspot mutations in codons 36, 43, and 44 (Makinen et al., 2011, Science, 334, 252-255; Kämpjärvi et al., 2014, Hum Mutat, 35, 1136-1141). Second, localization of the missense mutations to a small number of amino-acids suggests that the MED12 mutations are dominant and that MED12 acts as an oncogene (Vogelstein et al., 2013, Science, 339, 1546-1558), providing a likely etiological basis previously lacking for the majority of these clinically significant tumors. Compatible with the key role of MED12 in controlling gene expression, Mehine et al. also showed that the RNA expression patterns of MED12 mutant leiomyomas cluster tightly together and form a clearly separate branch distinct from all other leiomyomas (Mehine et al., 2013, N. Engl. J. Med., 369, 43-53).

There remains a need for additional compositions and methods for treating cancerous and precancerous conditions.

### SUMMARY

Certain embodiments are directed to a peptide comprising all or part of an amino acid sequence that is at least 90% identical to the amino acid sequence of MAAFGILSYEHRPLKRPRLGPPDVYPQDPKQKEDELTALNVKQGFNNQPAVSGDEH GSAKNVSFNPAKISSNFSSIIAEKLRCNTLPDT (SEQ ID NO:1). In certain aspects a peptide can comprise 10, 20, 30, 40, 50, 60, 70, 80, 90 or 100 consecutive amino acids that is 90, 95, or 100% identical SEQ ID NO:l. In certain embodiments a peptide described herein can be comprised in a pharmaceutical composition. Certain aspects are directed to an expression vector encoding a peptide as described herein.

Certain embodiments are directed to methods for treating a subject having a cancerous or precancerous condition comprising administering a peptide described herein to a subject having or suspected of having a cancerous or precancerous condition. In certain aspects the cancerous or precancerous condition is associated with amplification-dependent overexpression of CDK8, e.g., colorectal cancer. In certain aspects the condition to be treated is leiomyomas, the treatment comprising administering a peptide described herein to a subject having or suspected of having uterine leiomyomas.

Other embodiments are directed to modulating CDK8 activity by administering a peptide described herein to a cell in need of CDK8 kinase modulation. In certain aspects the peptide is an inhibitor of CDK8 kinase activity, which would be used to treat those cancers and hyperproliferative conditions associated with an increased CDK8 activity. In other aspects the polypeptide or fragment thereof is an activator of CDK8 kinase activity, which would be used to treat those conditions resulting from the lack or CDK8 kinase activation, e.g., treating uterine leiomyomas and the like.

The terms “treating” or “treatment” refer to any success or indicia of success in the attenuation or amelioration of an injury, pathology or condition, including any objective or subjective parameter such as abatement, remission, diminishing of symptoms or making the injury, pathology, or condition more tolerable to the patient, slowing in the rate of degeneration or decline, making the final point of degeneration less debilitating, improving a subject's physical or mental well-being, or prolonging the length of survival. The treatment or amelioration of symptoms can be based on objective or subjective parameters; including the results of a physical examination, neurological examination, and/or psychiatric evaluations.

The term “isolated” can refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated compound refers to one that can be administered to a subject as an isolated compound; in other words, the compound may not simply be considered “isolated” if it is adhered to a column or embedded in an agarose gel. Moreover, an “isolated nucleic acid fragment” or “isolated peptide” is a nucleic acid or protein fragment that is not naturally occurring as a fragment and/or is not typically in the functional state.

Moieties of the invention, such as polypeptides, peptides, antigens, or immunogens, may be conjugated or linked covalently or noncovalently to other moieties such as adjuvants, proteins, peptides, supports, fluorescence moieties, or labels. The term “conjugate” or “immunoconjugate” is broadly used to define the operative association of one moiety with another agent and is not intended to refer solely to any type of operative association, and is particularly not limited to chemical “conjugation.”

The term “providing” is used according to its ordinary meaning “to supply or furnish for use.” In some embodiments, the protein is provided directly by administering the protein, while in other embodiments, the protein is effectively provided by administration of a nucleic acid encoding the protein. In certain aspects the invention contemplates compositions comprising various combinations of nucleic acid, antigens, peptides, and/or epitopes.

The phrase “specifically binds” or “specifically immunoreactive” to a target refers to a binding reaction that is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane, Antibodies: A Laboratory Manual, Cold Spring Harbor Press, 1988, for a description of immunoassay formats and conditions that can be used to determine specific immunoreactivity.

“Effective amount” and “therapeutically effective amount” are used interchangeably herein, and refer to an amount of a nucleic acid, polypeptide, peptide, antibody, or functional fragment thereof, as described herein, effective to achieve a particular biological or therapeutic result such as, but not limited to, the biological or therapeutic results disclosed herein. A therapeutically effective amount of the nucleic acid, polypeptide, peptide, antibody, or functional fragment thereof may vary according to factors such as the disease state, age, sex, and weight of the individual, and the ability of the antibody or functional fragment thereof to elicit a desired response in the individual. Such results may include, but are not limited to, the treatment of cancer, as determined by any means suitable in the art.

“Substantially similar” with respect to nucleic acid or amino acid sequences, means at least about 65% identity between two or more sequences. Preferably, the term refers to at least about 70% identity between two or more sequences, more preferably at least about 75% identity, more preferably at least about 80% identity, more preferably at least about 85% identity, more preferably at least about 90% identity, more preferably at least about 91% identity, more preferably at least about 92% identity, more preferably at least about 93% identity, more preferably at least about 94% identity, more preferably at least about 95% identity, more preferably at least about 96% identity, more preferably at least about 97% identity, more preferably at least about 98% identity, and more preferably at least about 99% or greater identity. Such identity can be determined using algorithms known in the art, such as the BLAST algorithm.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Leiomyoma-Linked Mutations in MED12 Disrupt its Interaction with Cyclin C-CDK8/19 and Diminish Mediator-Associated Kinase Activity. (A) Schematic illustration of MED12 WT and G44D mutant protein-protein-interactome screen. (B) Normalized abundance of MED12WTand G44D-interaction proteins. Modular arrangement of the Mediator is shown. Note that MED12 mutation G44D results in specific decrease in binding of kinase module subunits. (C) Average change of binding of MED12 G44D mutant to different Mediator modules. Data represent the average±SD of three independent experiments. The only statistically significant change was found with the kinase module (p=2×10⁻⁵. For the other modules, p>0.2. See Table 2 for description of statistical analysis. (D) Immunoprecipitation (IP)-western blot (WB) verification of the loss of CDK8/CDK19 binding to the G44D mutant MED12 from 293 Flp-In cells. (E) FLAG-tagged WT MED12 or its indicated mutant derivatives were immunoprecipitated from transfected HEK293 cell lysates. FLAG-specific immunoprecipitates were processed by WB using the indicated antibodies (middle panel) or incubated with [g-32P]-ATP and purified glutathione S-transferase (GST)-CTD (bottom). INPUT (top) corresponds to 10% of cell lysate used in IP reactions. 32P-GST-CTD levels were quantified and expressed relative to the level in the WT MED12 IP. Data represent the average±SEM of three independent experiments. Asterisks denote statistically significant differences versus WT (Student's t test, ***p<0.001). Note that the CDK19 WB was derived from the same IP but a different gel to preclude interference from the signal produced by similarly sized CDK8. (F) Baculovirus-expressed FLAG-CDK8, Cyclin C-H6, and MED12-HA (WT or mutant as indicated) were immunoprecipitated from infected insect cell lysates. FLAG-specific immunoprecipitates were processed by WB using the indicated antibodies (top) or subjected to in vitro kinase assay (bottom) as in (E). Input corresponds to 10% of cell lysate used in IP reactions. WBs were quantified and levels of MED12 and Cyclin C (CycC) in each IP were normalized to CDK8 levels and expressed relative to their corresponding normalized levels in the CDK8/CycC/MED12 WT IP (middle). 32P-GST-CTD levels in each IP/kinase reaction were quantified and expressed relative to the level in the CDK8/CycC/MED12 WT IP. Data represent the average±SEM of three independent experiments. Asterisks denote statistically significant differences versus WT (Student's t test, *p<0.05, ***p<0.001).

FIG. 2. The MED12 N-Terminal 100 Amino Acids Bind to and Activates Cyclin C-CDK8. (A) Glutathione S-transferase (GST)-MED12 fragments as indicated were immobilized on glutathione-Sepharose and incubated with insect cell lysates coexpressing Cyclin C/CDK8. Bound proteins were eluted with Laemmli sample buffer and resolved by SDS-10% PAGE prior to visualization by Coomassie blue staining (left) or WB analysis (right) using antibodies specific for CDK8 and Cyclin C. INPUT, 10% of insect cell lysate used in GST pull-down reactions. (B) FLAG-specific immunoprecipitates from insect cells infected without (FLAG-Control) or with (FLAG-CDK8/CycC) baculoviruses expressing FLAG-CDK8 and CycCH6 were incubated with the indicated ³⁵5-labeled MED12 truncation fragments produced by transcription and translation in vitro. Bound proteins were eluted with Laemmli sample buffer and resolved by SDS-12% PAGE prior to visualization by Phosphorimager analysis. INPUT, 10% of the in vitro translated MED12 protein fragments used in binding reactions. Phosphorimager signals were quantified, and the level of binding for each MED12 fragment to CDK8/Cyclin C is expressed relative to the 10% INPUT signal. (C) (Left) Purified HA-MED12 (1-100) bearing tandem six-histidine and hemagglutinin (HA)-epitope tags was expressed in E. coli and purified on nickel-nitrilotriacetic acid prior to resolution by SDS-15% PAGE and visualization by Coomassie blue staining. Molecular weight marker positions (kDa) are indicated. (Right) Baculovirus-expressed Cyclin C-H6/FLAG-CDK8 was immunoprecipitated from infected insect cell lysates in the absence (-31 ) or presence (+) of HA-MED12 (1-100). FLAG-specific immunoprecipitates were processed by western blot using the indicated antibodies (top) or incubated with [γ-³²P]-ATP and purified GST-CTD (bottom). INPUT corresponds to 5% of protein used in IP reactions. ³²P-GST-CTD levels were quantified and expressed relative to the level in the absence of MED12 (1-100). Data represent the average±SEM of three independent experiments. p value calculated by Student's t test.

FIG. 3. The cyclin C surface groove is required for MED12 binding and CDK8 activation. (A) Schematic summary of binding interactions within the Mediator kinase module. (B) Structure of H. sapiens Cyclin C-CDK8 (25) (Protein Data Bank accession number 3RGF). Cyclin C, blue; CDK8, gray. Targeted residues that lie within (W177, N181, D182, and Y238) and outside (W6 and E98) the groove are rendered yellow. (C) Baculovirus-expressed MED12-HA, FLAG-CDK8, and CycC-H6 (WT or mutant as indicated) were immunoprecipitated from infected insect cell lysates. FLAG-specific immunoprecipitates were processed by western blot (WB) using the indicated antibodies (top) or incubated with [γ-³²P]-ATP and purified glutathione S-transferase-CTD (bottom). Input (IN) corresponds to 10% of cell lysate used in IP reactions. WBs and kinase reactions were quantified, and binding and kinase levels calculated as described in the legend to FIG. 1F. Data represent the average±SEM of three independent experiments. Asterisks denote statistically significant differences versus WT (Student's t test, **p<0.01, ***p<0.001). (D) FLAG-tagged WT Cyclin C or its indicated mutant derivatives were immunoprecipitated from transfected HEK293 cell lysates. FLAG-specific immunoprecipitates were processed by WB using the indicated antibodies (top) or subjected to in vitro kinase assay as in (C). INPUT corresponds to 10% of cell lysate used in IP reactions. ³²P-GST-CTD levels were quantified and expressed relative to the level in the WT Cyclin C IP.

FIG. 4. Identification of subunit interactions within the Mediator kinase module. (A and B) Lysates from Insect cells expressing the indicated combinations of CBP-MED13, MED12-HA, FLAG-CDK8, or Cyclin C 6-His (CycCH6), were subjected to IP using antibodies specific for HA (A) or CBP (B). Immunoprecipitates were resolved by SDS-10% PAGE and subjected to WB analysis using indicated antibodies. Input, 10% of lysates used for IP reactions. (C) GST or GSTMED12 (1-330) as indicated were immobilized on glutathione-Sepharose and incubated with insect cell lysates expressing Cyclin C, CDK8, or both. Bound proteins were eluted with Laemmli sample buffer and resolved by SDS-10% PAGE prior to visualization by Coomasie Blue staining (left panel) or WB analysis (right panel) using antibodies specific for CDK8 and Cyclin C. INPUT, 10% of insect cell lysate used in GST pull-down reactions.

FIG. 5. Cyclin C surface groove mutant N181A is excluded from high-molecular mass Mediator complex. (A and B) Nuclear extracts from HEK 293 cells transfected with FLAG Cyclin C WT (A) or its N181A mutant derivative (B) were fractionated on Superose 6 gel filtration columns. The indicated column fractions (2% column volume each) were resolved by SDS-10% PAGE and analyzed by WB using antibodies specific for the FLAG epitope on Cyclin C or individual Mediator subunits as indicated. The positions of molecular mass standards are indicated. Note that ectopically expressed Cyclin C surface groove mutant N181A, but not WT Cyclin C, is excluded from a high molecular mass (˜2 MDa) Mediator complex. The presence of CDK8 in high molecular mass fractions in both extracts is due to the presence of endogenous Cyclin C.

### DESCRIPTION

Mediator is a conserved multisubunit signal processor through which regulatory information conveyed by gene-specific transcription factors is transduced to RNA polymerase II (pol II). Structurally, Mediator is assembled from a set of core subunits into three distinct modules termed “head”, “middle”, and “tail”, that bind tightly to pol II in the so-called holoenzyme (Conaway and Conaway, 2011, Curr. Opin. Genet. Dev., 21, 225-230; Kornberg, 2005, Trends Biochem. Sci., 30, 235-239; Larivière et al., 2012, Curr. Opin. Cell Biol., 24, 305-313; Malik and Roeder, 2010, Nat. Rev. Genet., 11, 761-772; Spaeth et al., 2011, Cell Dev. Biol., 22, 776-787; Taatjes, 2010, Trends Biochem. Sci., 35, 315-322). MED12, along with MED13, Cyclin C and CDK8 or CDK19, comprise a fourth “kinase” module that exists in variable association with core Mediator. The kinase module was originally implicated in negative regulation of pol II dependent transcription (Akoulitchev et al., 2000, Nature, 407, 102-106; Knuesel et al., 2009, Genes Dev., 23, 439-451; van de Peppel et al., 2005, Mol. Cell, 19, 511-522). Several recent studies, however, have also characterized a positive role for CDK8 activity in transcription (Donner et al., 2010, Nat. Struct. Mol. Biol., 17, 194-201; Firestein et al., 2008, Nature, 455, 547-551; Morris et al., 2008, Nature, 455, 552-556).

MED12 links Cyclin C-CDK8 with core Mediator and also stimulates Cyclin C-dependent CDK8 kinase activity (Ding et al., 2008, Mol. Cell, 31, 347-359; Knuesel et al., 2009, Mol. Cell. Biol., 29, 650-661). Although the mechanism by which MED12 activates CDK8 is unknown, MED12-dependent CDK8 activation is nonetheless required for nuclear transduction of signals propagated by several different oncogenic pathways with which MED12 is biochemically and genetically linked (Firestein et al., 2008, Nature, 455, 547-551; Kim et al., 2006, J. Biol. Chem., 281, 14066-14075; Spaeth et al., 2011, Cell Dev. Biol., 22, 776-787; Zhou et al., 2006, Mol. Cell. Biol., 26, 8667-8682; Zhou et al., 2012, Proc. Natl. Acad. Sci. USA, 109, 19763-19768). Furthermore, MED12 itself is a target of oncogenic mutation, including exon 1 and 2 mutations linked to uterine leiomyomas (Barbieri et al., 2012, Nat. Genet., 44, 685-689; Je et al., 2012, Int. J. Cancer, 131, E1044-E1047; Kämpjärvi et al., 2012, Br. J. Cancer, 107, 1761-1765; Makinen et al., 2011, Science, 334, 252-255; Kämpjärvi et al., 2014, Hum Mutat, 35, 1136-1141). However, the impact of these mutations on MED12 function and the molecular basis for their tumorigenic potential remain unknown.

### I. POLYPEPTIDE COMPOSITIONS

Modifications and/or changes may be made in the amino acid composition of peptides described herein, and thus variation in sequences of the peptides and nucleic acids coding them is contemplated, such that the variant peptides substantially retain the activity with respect to the therapeutic, preventative, and curative aspects of the present invention.

In one example, a polynucleotide may encode a peptide having a biological functional equivalent. Certain amino acids may be substituted for other amino acids in a peptide without appreciable loss of interactive binding capacity and activity. So-called “conservative” changes do not disrupt the biological activity of the peptide, as the change is not one that impinges on the peptide's ability to carry out its designed function. It is thus contemplated by the inventors that various changes may be made in the sequence of polynucleotides and peptides disclosed herein.

In terms of functional equivalents, it is well understood by the skilled artisan that, inherent in the definition of a “biologically functional equivalent” peptide and/or polynucleotide, is the concept that there is a limit to the number of changes that may be made within a defined portion of the molecule while retaining a molecule with an acceptable level of equivalent biological activity. Biologically functional equivalents are thus defined herein as those peptides (and polynucleotides) in which selected amino acids (or nucleotides) may be substituted. In certain aspects, a polypeptide is 80, 85, 90, 92, 94, 96, 98, or 100% identical to all or part of SEQ ID NO:l.

In general, the shorter the length of the molecule, the fewer changes that can be made within the molecule while retaining function. Longer domains may have an intermediate number of changes. The full-length protein will have the most tolerance for a larger number of changes. However, it must be appreciated that certain molecules or domains that are highly dependent upon their structure may tolerate little or no modification. Function of a polypeptide can be determined by using various assays know to detect the activity of the polypeptide of interest.

Amino acid substitutions are generally based on the relative similarity of the amino acid side-chain substituents, for example, their hydrophobicity, hydrophilicity, charge, size, and/or the like. An analysis of the size, shape and/or type of the amino acid side-chain substituents reveals that arginine, lysine, and/or histidine are all positively charged residues; that alanine, glycine, and/or serine are all a similar size; and/or that phenylalanine, tryptophan, and/or tyrosine all have a generally similar shape. Therefore, based upon these considerations, arginine, lysine, and/or histidine; alanine, glycine, and/or serine; and/or phenylalanine, tryptophan, and/or tyrosine are defined herein as biologically functional equivalents.

To effect more quantitative changes, the hydropathic index of amino acids may be considered. Each amino acid has been assigned a hydropathic index on the basis of their hydrophobicity and/or charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and/or arginine (−4.5).

The importance of the hydropathic amino acid index in conferring interactive biological function on a protein is generally understood in the art (Kyte & Doolittle, 1982, incorporated herein by reference). It is known that certain amino acids may be substituted for other amino acids having a similar hydropathic index and/or score and/or still retain a similar biological activity. In making changes based upon the hydropathic index, the substitution of amino acids whose hydropathic indices are within ±2 is preferred, those that are within ±1 are particularly preferred, and/or those within ±0.5 are even more particularly preferred.

It also is understood in the art that the substitution of like amino acids can be made effectively on the basis of hydrophilicity. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those that are within ±1 are particularly preferred, and/or those within ±0.5 are even more particularly preferred.

The term “nucleic acid vector” is used to refer to a carrier nucleic acid molecule into which a nucleic acid sequence can be inserted for introduction into a cell where it can be replicated, transcribed, and/or translated (i.e., expressed). A nucleic acid sequence can be “exogenous,” which means that it is foreign to the cell into which the vector is being introduced or that the sequence is “endogenous” to the cell but in a position within the host cell in which the sequence is ordinarily not found. In certain aspects an exogenous vector can encode an endogenous nucleic acid. Nucleic acid vectors include plasmids, cosmids, viral genomes, and other expression vectors (bacteriophage, animal viruses, and plant viruses), artificial chromosomes (e.g., YACs), and the like. Given the current disclosure, one of skill in the art would be well equipped to construct a vector through standard recombinant techniques (see, for example, Maniatis et al., Molecular Cloning: A laboratory Manual. Cold Spring Harbor Laboratory, New York., 1989; Ausubel et al., Current Protocols in Molecular Biology, New York City, N.Y., John Wiley & Sons, Inc., 1994, both incorporated herein by reference).

The term “expression vector” refers to any type of genetic construct comprising a nucleic acid coding for an RNA capable of being transcribed. In some cases, RNA molecules are then translated into a protein, polypeptide, or peptide. In other cases, these sequences are not translated, for example, in the production of inhibitory RNA, antisense molecules, or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described herein.

A. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. Additional promoter elements regulate the frequency of transcriptional initiation. Typically, these are located in the region 30-110 by upstream of the start site, although a number of promoters have been shown to contain functional elements downstream of the start site as well. To bring a coding sequence “under the control of” a promoter, one positions the 5′ end of the transcription initiation site of the transcriptional reading frame “ downstream” of (i.e., 3′ of) the chosen promoter. The “upstream” promoter stimulates transcription of the DNA and promotes expression of the RNA. The spacing between promoter elements frequently is flexible, so that promoter function is preserved when elements are inverted or moved relative to one another. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous” or “homologous.” Similarly, an enhancer may be one naturally associated with a nucleic acid sequence, located either downstream or upstream of that sequence. Alternatively, certain advantages will be gained by positioning the nucleic acid under the control of a recombinant, exogenous, or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from another virus, or prokaryotic or eukaryotic cell, and promoters or enhancers not “naturally occurring,” i.e., containing different elements of different transcriptional regulatory regions, and/or mutations that alter expression.

Naturally, it will be important to employ a promoter and/or enhancer that effectively directs the expression of the DNA segment in the organelle, cell, tissue, organ, or organism chosen for expression. Those of skill in the art of molecular biology generally know the use of promoters, enhancers, and cell type combinations for protein expression, (see, for example Sambrook et al., Molecular Cloning: A Laboratory Manual, vol. I. 2nd edition. Cold Spring Harbor Laboratory Press, 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, world-wide-web at epd.isb-sib.ch/) could also be used to drive expression. Use of a T3, T7, or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

In certain aspects, a nucleic acid of the invention can comprise a non-inducible or inducible promoter that will be expressed specifically in the pancreatic tissues. Such non-inducible promoters include tissue-specific pancreas promoters from the insulin gene, glucagon gene, amylase gene, etc. Such inducible promoters include pancreas specific promoters under the control of the glucose response element or pancreas specific promoter under the control of a response element that is inducible by chemical, peptide, ligand, or metabolites.

B. Initiation Signals

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

C. Multiple Cloning Sites

Vectors can include a multiple cloning site (MCS), which is a nucleic acid region that contains multiple restriction enzyme sites, any of which can be used in conjunction with standard recombinant technology to digest the vector. “Restriction enzyme digestion” refers to catalytic cleavage of a nucleic acid molecule with an enzyme that function only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

D. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

Terminators contemplated for use in the invention include any known terminator of transcription described herein or known to one of ordinary skill in the art, including but not limited to, the termination sequences such as bovine growth hormone terminator or viral termination sequences, such as the SV40 terminator. In certain embodiments, the termination signal may be a lack of transcribable or translatable sequence, such as due to a sequence truncation.

E. Post-Transcriptional Regulatory Elements (PRE)

Post-transcriptional regulation is the control of gene expression at the RNA level, i.e., between the transcription and the translation of the gene. In certain aspects, the Woodchuck Hepatitis Virus Post-transcriptional Regulatory Element (WPRE) is used. WPRE increases the levels of nuclear transcripts and facilitates RNA export. WPRE may facilitate other steps in RNA processing, directing RNAs that would normally be degraded within the nucleus to be efficiently expressed. The WPRE can also function to facilitate the generation of RNA-protein complexes that would protect newly synthesized transcripts from degradation in the nucleus. (Zufferey et al., Journal of Virology, 73: 2886-2892, 1999 and U.S. Pat. No. 6,284,469, which is incorporated herein by reference).

F. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

G. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

H. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with fluorescence assisted cell sorting (FACS) and/or immunohistochemistry. The marker used is not believed to be important, so long as it is capable of being expressed simultaneously with the nucleic acid encoding a gene product. Further examples of selectable and screenable markers are well known to one of skill in the art.

In certain aspects, components are provided to a uterus or other organ or tissue by using nucleic acids that encode or express such components. Viral and non-viral delivery vectors can be used in the methods described herein. The term “nucleic acids”, “nucleic acid molecules”, “nucleic acid sequences”, “nucleotide sequences” and “nucleotide molecules” are used interchangeably herein and, unless otherwise specified, refer to a polymer of deoxyribonucleic acids, including cDNA, DNA, PNA, or polymers of ribonucleic acids (RNA). Nucleic acid may be obtained from a cellular extract, genomic or extragenomic DNA, viral nucleic acids, or artificially/chemically synthesized molecules. The term can include double stranded or single stranded deoxyribonucleic or ribonucleic acids.

Viral delivery. The ability of certain viruses to infect cells or enter cells via receptor-mediated endocytosis, and to express virally encoded genes have made them attractive candidates for the transfer of foreign nucleic acids into cells (e.g., mammalian cells). Viruses may thus be utilized that encode and express agents to increase the activity of glucose metabolism, increase tyrosine kinase receptor activity, and increase transcription of genes associated with beta cells. Non-limiting examples of virus vectors that may be used to deliver nucleic acids are described below.

Adenoviral Vectors. A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, Semin. Virol. 3, 237-252, 1992).

AAV Vectors. The nucleic acid may be introduced into the cell using adenovirus-assisted transfection. Increased transfection efficiencies have been reported in cell systems using adenovirus-coupled systems. Adeno-associated virus (AAV) has a low frequency of integration and it can infect non-dividing cells, thus making it useful for delivery of genes into mammalian cells in tissue culture or in vivo. AAV has a broad host range for infectivity. Details concerning the generation and use of rAAV vectors are described in U.S. Pat. Nos. 5,139,941 and 4,797,368, each of which incorporated herein by reference.

Retroviral Vectors. Retroviruses have the ability to integrate their genes into the host genome, transferring a large amount of foreign genetic material, infecting a broad spectrum of species and cell types and of being packaged in special cell-lines. In order to construct a retroviral vector, a nucleic acid (e.g., one encoding a protein of interest) is inserted into the viral genome in the place of certain viral sequences to produce a virus that is replication-defective. In order to produce virions, a packaging cell line containing the gag, pol, and env genes but without the LTR and packaging components is constructed. When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types.

Lentiviruses are complex retroviruses, which, in addition to the common retroviral genes gag, pol, and env, contain other genes with regulatory or structural function. Lentiviral vectors are well known in the art (see, for example, U.S. Pat. Nos. 6,013,516 and 5,994,136, each of which is incorporated herein by reference). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and that is described in U.S. Pat. No. 5,994,136, which is incorporated herein by reference.

One may target the recombinant virus by linkage of an envelope protein with an antibody or a particular ligand for targeting to a receptor of a particular cell-type. By inserting a sequence (including a regulatory region) of interest into the viral vector, along with another gene that encodes the ligand for a receptor on a specific target cell, for example, the vector is now target-specific. Such viral vectors can be targeted to cells of the pancreas.

Other Viral Vectors. Other viral vectors may be employed in the methods of the present invention. Vectors derived from viruses such as vaccinia virus (Ridgeway, In: Vectors: A survey of molecular cloning vectors and their uses, Rodriguez and Denhardt (Eds.), Stoneham: Butterworth, 467-492, 1988; Baichwal and Sugden, In: Gene Transfer, Kucherlapati (Ed.), NY, Plenum Press, 117-148, 1986; Coupar et al., Gene 68:1-10, 1988), sindbis virus, cytomegalovirus, and herpes simplex virus may be employed. They offer several attractive features for various mammalian cells (Friedmann, Science, 244:1275-1281, 1989; Ridgeway, In: Vectors: A survey of molecular cloning vectors and their uses, Rodriguez and Denhardt (Eds.), Stoneham: Butterworth, 467-492, 1988; Baichwal and Sugden, In: Gene Transfer, Kucherlapati (Ed.), NY, Plenum Press, 117-148, 1986; Coupar et al., Gene 68:1-10, 1988; Horwich et al., J. Virol. 64:642-650, 1990).

### II. PHARMACEUTICAL COMPOSITIONS

In light of the current specification, the determination of an appropriate treatment regimen (e.g., dosage, frequency of administration, systemic vs. local, etc.) is within the skill of the art. For administration, the components described herein will be formulated in a unit dosage form (solution, suspension, emulsion, etc.) in association with a pharmaceutically acceptable carrier. Such vehicles are usually nontoxic and non-therapeutic. Examples of such vehicles are water, saline, Ringer's solution, dextrose solution, and Hank's solution. Non-aqueous vehicles such as fixed oils and ethyl oleate may also be used. A preferred vehicle is 5% (w/w) human albumin in saline. The vehicle may contain minor amounts of additives, such as substances that enhance isotonicity and chemical stability, e.g., buffers and preservatives.

The therapeutic compositions described herein, as well as their biological equivalents, can be administered independently or in combination by any suitable route. Examples of parenteral administration include intravenous, intraarterial, intramuscular, intraperitoneal, and the like. The routes of administration described herein are merely an example and in no way limiting.

The dose of the therapeutic compositions administered to an animal, particularly in a human, in accordance with embodiments of the invention, should be sufficient to result in a desired response in the subject over a reasonable time frame. It is known that the dosage of therapeutic compositions depends upon a variety of factors, including the strength of the particular therapeutic composition employed, the age, species, condition or disease state, and the body weight of the animal.

Moreover, dose and dosage regimen, will depend mainly on the type of biological damage to the host, the type of subject, the history of the subject, and the type of therapeutic composition being administered. The size of the dose will be determined by the route, timing and frequency of administration as well as the existence, nature and extent of any adverse side effects that might accompany the administration of a particular therapeutic composition and the desired physiological effect. It is also known that various conditions or disease states, in particular, chronic conditions or disease states, may require prolonged treatment involving multiple administrations.

Therefore, the amount of the therapeutic composition must be effective to achieve an enhanced therapeutic index. If multiple doses are employed, the frequency of administration will depend, for example, on the type of subject. One skilled in the art can ascertain upon routine experimentation the appropriate route and frequency of administration in a given subject that are most effective in any particular case. Suitable doses and dosage regimens can be determined by conventionally known range-finding techniques. Generally, treatment is initiated with smaller dosages, which are less than the optimal dose of the compound. Thereafter, the dosage is increased by small increments until the optimal effect under the circumstances is obtained.

The therapeutic compositions for use in embodiments of the invention generally include carriers. These carriers may be any of those conventionally used and are limited only by the route of administration and chemical and physical considerations, such as solubility and reactivity with the therapeutic agent. In addition, the therapeutic composition may be formulated as polymeric compositions, inclusion complexes, such as cyclodextrin inclusion complexes, liposomes, microspheres, microcapsules, and the like, without limitation.

The pharmaceutically acceptable excipients described herein, for example, vehicles, adjuvants, carriers, or diluents, are well known and readily available. It is preferred that the pharmaceutically acceptable carrier be one which is chemically inert with respect to the therapeutic composition and one that has no detrimental side effects or toxicity under the conditions of use.

The choice of excipient will be determined, in part, by the particular therapeutic composition, as well as by the particular method used to administer the composition. Accordingly, there are a wide variety of suitable formulations of the pharmaceutical composition used in the embodiments of the invention. For example, the non-limiting formulations can be injectable formulations such as, but not limited to, those for intravenous, subcutaneous, intramuscular, intraperitoneal injection, and the like, and oral formulations such as, but not limited to, liquid solutions, including suspensions and emulsions, capsules, sachets, tablets, lozenges, and the like. Non-limiting formulations suitable for parenteral administration include aqueous and non-aqueous isotonic sterile injection solutions, including non-active ingredients such as antioxidants, buffers, bacteriostats, solubilizers, thickening agents, stabilizers, preservatives, surfactants, and the like. The solutions can include oils, fatty acids, including detergents and the like, as well as other well-known and common ingredients in such compositions, without limitation.

### III. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

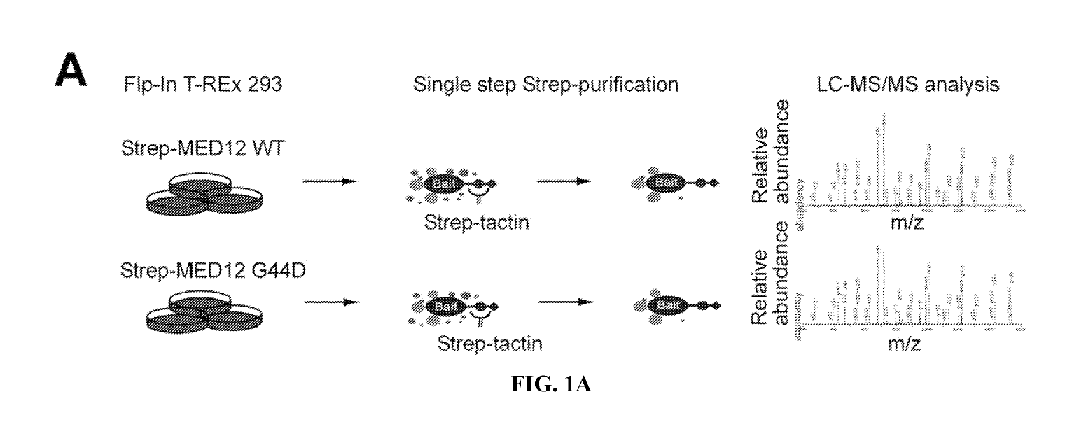

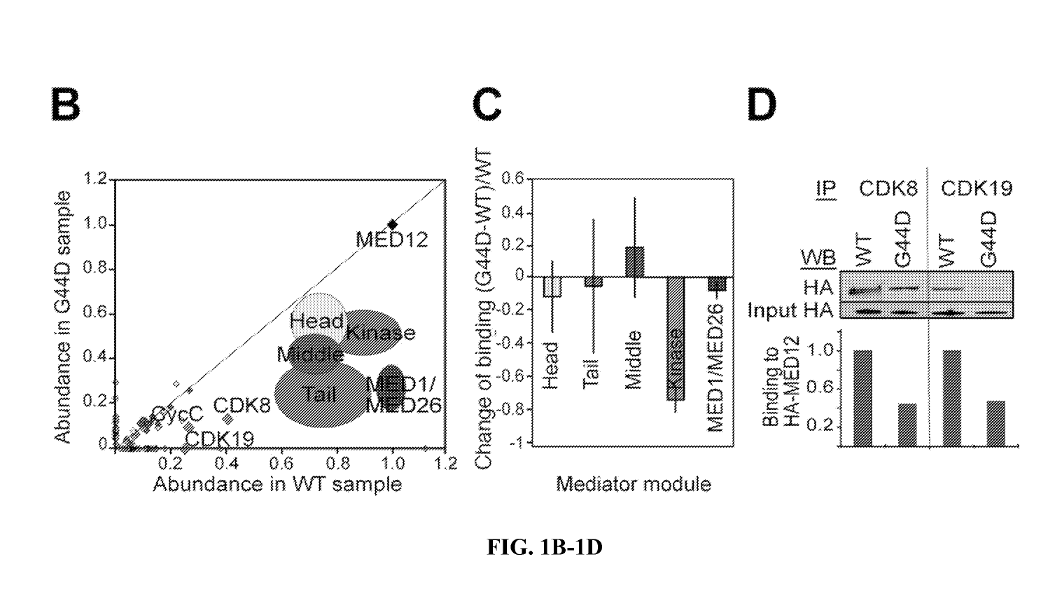

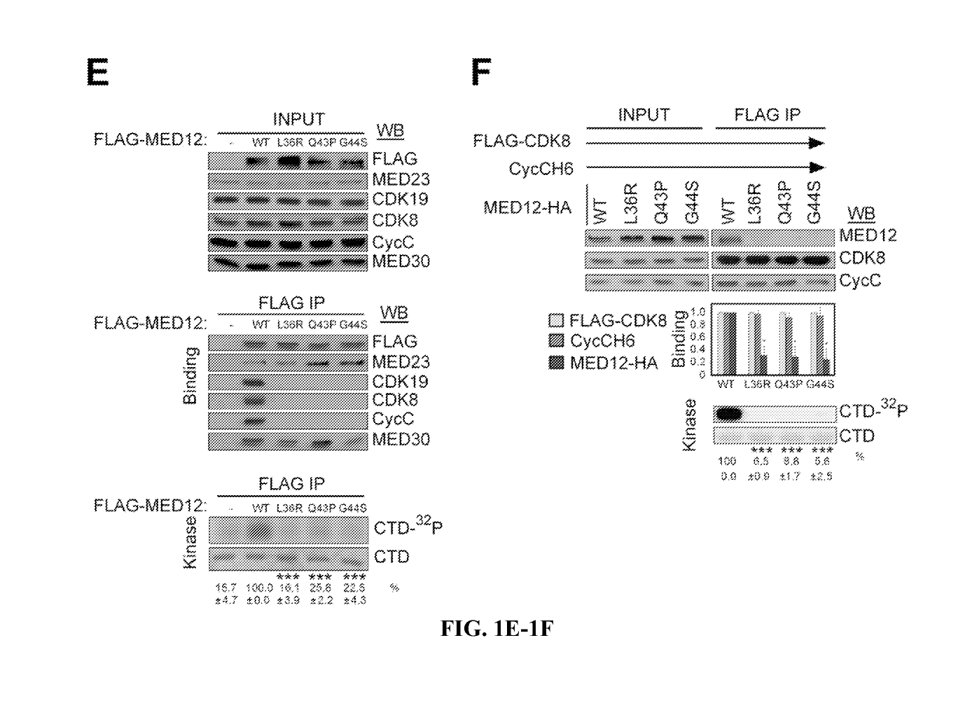

Uterine leiomyoma-linked mutations in MED12 disrupt its association with CycC-CDK8/19. To identify proteins that bind differentially to wild-type and oncogenic MED12, stable, tetracycline inducible Flp-In™ M 293 T-REx cell lines expressing C terminally Twin-Strep-tagR-modified wild-type (WT) MED12 or its most common leiomyoma mutant derivative (G44D) was engineered (Glatter et al., 2009, Mol. Syst. Biol., 5, 237; Varjosalo et al., 2013, Cell Rep., 3, 1306-1320)). Quantitative immunoblot analysis revealed that tagged WT and mutant MED12 proteins attained induced levels of expression (˜0.8-1.6×10⁵ molecules per cell) comparable to that of endogenous 293 cell MED12 (˜0.4-0.8×10⁵ molecules per cell). Affinity-purification mass spectrometry (AP-MS) (FIG. 1A) revealed a specific and reproducible (n =3) reduction in the binding of Cyclin C, CDK8, and CDK19 to mutant versus wild-type MED12 (FIG. 1B; Table 1). Relative quantification of MED12-associated Mediator subunits confirmed a statistically significant loss of kinase module, as opposed to core, Mediator subunits in mutant versus wild-type MED12 affinity purifications (FIG. 1C and Table 2). The reduced association of CDK8 and CDK19 with MED12 G44D was confirmed by IP-Western (FIG. 1D), and it was further established that this defect extends to other uterine leiomyoma-linked exon 2 mutations in MED12, including L36R, Q43P, and G44S. Thus, FLAG-specific immunoprecipitates from HEK293 cells expressing FLAG-tagged MED12 mutant derivatives bore significantly reduced levels of Cyclin C, CDK8, and CDK19, but not core Mediator subunits, as well as diminished pol II C-terminal domain (CTD)-directed kinase activity compared to those from wild-type MED12-expressing cells (FIG. 1E).

TABLE 1
Normalized affinity purification LC-MS results. List of normalized
abundance of proteins calculated with Spectral counting from LC-MS
after the affinity purifications of WT MED12 and G44D MED12
(Zybailov et al., 2005) (Paoletti et al., 2006) (Liu et al., 2004). Each
average and STDEV was calculated from 3 replicate experiments.
Normalization was done by dividing the peptide spectrum match
(PSM) values for each protein with protein length and amount of
bait protein detected in the sample.

| | Avg. | Avg. | STDEV | STDEV |
|---|---|---|---|---|
| | Normalized | Normalized | Normalized | Normalized |
| | abundance | abundance | abundance | abundance |
| Gene Name | WT | G44D | WT | G44D |
| HIST1H1D | 1.117736241 | 0 | 0.111912709 | 0 |
| MED12 | 1 | 1 | 0 | 0 |
| CDK8 | 0.404019639 | 0.131504102 | 0.035748789 | 0.056603783 |
| SEC61G | 0.37781987 | 0 | 0.063533742 | 0 |
| MED4 | 0.266297607 | 0.260405668 | 0.04118066 | 0.043935023 |
| CCNC | 0.263550036 | 0.094935953 | 0.018910915 | 0.04892378 |
| CDK19 | 0.250683435 | 0 | 0.082810041 | 0 |
| MED6 | 0.239963931 | 0.125696189 | 0.063281189 | 0.018278654 |
| MED22 | 0.218088509 | 0.288940686 | 0.077864983 | 0.071801034 |
| MED27 | 0.198499362 | 0.183885452 | 0.049913975 | 0.07948882 |
| AGXT | 0.177349577 | 0 | 0.053624082 | 0 |
| MED11 | 0.176919644 | 0.163262322 | 0.153252684 | 0.091138347 |
| MED17 | 0.173296285 | 0.159908288 | 0.038941457 | 0.004196346 |
| MED30 | 0.172945009 | 0.167736759 | 0.04146452 | 0.103598823 |
| MED20 | 0.157176922 | 0.099099701 | 0.039809059 | 0.026205197 |
| MED8 | 0.15304416 | 0.093963644 | 0.041673731 | 0.04411343 |
| MED16 | 0.152073511 | 0.112590982 | 0.031203657 | 0.026680287 |
| DDX55 | 0.145177103 | 0 | 0.05136236 | 0 |
| ILK | 0.142294912 | 0 | 0.012301254 | 0 |
| MED12L | 0.135511957 | 0.135641115 | 0.009824671 | 0.01608202 |
| MED10 | 0.133049535 | 0.143283131 | 0.031299486 | 0.044444136 |
| CNTLN | 0.131516588 | 0 | 0.017713299 | 0 |
| MED1 | 0.125131423 | 0.115151324 | 0.005802608 | 0.010348216 |
| SERPINC1 | 0.122094412 | 0 | 0.035806454 | 0 |
| ZSCAN4 | 0.117567518 | 0 | 0.103229601 | 0 |
| MED14 | 0.116638767 | 0.103308367 | 0.022676472 | 0.032620521 |
| KIAA1009 | 0.115292582 | 0 | 0.017666612 | 0 |
| ZBTB46 | 0.113584548 | 0 | 0.030813804 | 0 |
| GOSR1 | 0.111926918 | 0 | 0.106970132 | 0 |
| MED15 | 0.11050435 | 0.081358164 | 0.03550989 | 0.014929704 |
| GAL3ST2 | 0.110420751 | 0.122106963 | 0.058284118 | 0.042472243 |
| POLR2A | 0.108461608 | 0 | 0.017810259 | 0 |
| SGCD | 0.106396107 | 0 | 0.044729538 | 0 |
| TAOK1 | 0.105299309 | 0 | 0.023435753 | 0 |
| MED29 | 0.101938549 | 0.126515111 | 0.09640319 | 0.040140098 |
| MED18 | 0.099431394 | 0.09241566 | 0.05807673 | 0.026763785 |
| MED24 | 0.098688646 | 0.104285377 | 0.015367877 | 0.012611338 |
| MED31 | 0.097252558 | 0.129425564 | 0.089295376 | 0.070222898 |
| MED9 | 0.087260856 | 0.112833698 | 0.080121194 | 0.019576462 |
| GEMIN2 | 0.083744555 | 0 | 0.101397593 | 0 |
| AHNAK2 | 0.077427177 | 0.065123707 | 0.018117551 | 0.008979359 |
| KIAA0317 | 0.074966665 | 0 | 0.001264692 | 0 |
| MID1 | 0.07375387 | 0.07159707 | 0.064277068 | 0.018434266 |
| AKD1 | 0.065855566 | 0.052532444 | 0.005210315 | 0.008767698 |
| MED23 | 0.065547024 | 0.052878466 | 0.024742541 | 0.011266507 |
| NCAPG | 0.063077123 | 0 | 0.021099611 | 0 |
| MED19 | 0.063023314 | 0.077790681 | 0.054250636 | 0.069083836 |
| LIPE | 0.062269151 | 0 | 0.016011292 | 0 |
| ZZEF1 | 0.061883433 | 0 | 0.025351759 | 0 |
| TBX3 | 0.058815802 | 0 | 0.015746594 | 0 |
| EPB41L1 | 0.058397359 | 0 | 0.005730466 | 0 |
| CSE1L | 0.058295679 | 0.023247157 | 0.005594189 | 0.016645134 |
| ARAP2 | 0.052938279 | 0 | 0.045856796 | 0 |
| PCF11 | 0.052882498 | 0 | 0.005489986 | 0 |
| MARVELD3 | 0.0510751 | 0 | 0.039268734 | 0 |
| SLC22A16 | 0.049066701 | 0 | 0.008246102 | 0 |
| ITPR2 | 0.048763568 | 0 | 0.043043686 | 0 |
| MED13 | 0.048340717 | 0.025188804 | 0.015477104 | 0.023840924 |
| MED25 | 0.048114114 | 0.020926944 | 0.005225225 | 0.022990413 |
| EML1 | 0.047169083 | 0 | 0.024413456 | 0 |
| FMNL2 | 0.045150108 | 0 | 0.039375863 | 0 |
| GATA5 | 0.044873242 | 0 | 0.039978363 | 0 |
| TCERG1L | 0.043934055 | 0 | 0.045895607 | 0 |
| MED13L | 0.039513459 | 0.042400501 | 0.00496835 | 0.012351088 |
| N4BP2 | 0.037402251 | 0.031279259 | 0.032391898 | 0.027951419 |
| TNKS | 0.032931531 | 0 | 0.008816669 | 0 |
| RELN | 0.032616075 | 0 | 0.00672172 | 0 |
| CSMD3 | 0.027090214 | 0 | 0.004610666 | 0 |
| PTCH2 | 0.01694739 | 0 | 0.016027131 | 0 |
| CUL5 | 0 | 0.143131605 | 0 | 0.050915372 |
| IKBKAP | 0 | 0.060407798 | 0 | 0.002086239 |
| ITLN2 | 0 | 0.121566257 | 0 | 0.032062446 |
| KIF1C | 0 | 0.095034321 | 0 | 0.01188585 |
| KIF21B | 0 | 0.045292074 | 0 | 0.012103394 |
| KIF3A | 0 | 0.130498768 | 0 | 0.033420621 |
| MTHFR | 0 | 0.064969615 | 0 | 0.017035608 |
| GPR128 | 0 | 0.081171046 | 0 | 0.029618661 |
| FAT1 | 0 | 0.020769333 | 0 | 0.00848562 |
| EEF1A1P5 | 0 | 0.225586713 | 0 | 0.014373744 |
| SETBP1 | 0 | 0.075710684 | 0 | 0.021024169 |
| THAP11 | 0 | 0.298105349 | 0 | 0.06033301 |
| TSTD2 | 0 | 0.092318824 | 0 | 0.028293193 |
| KIAA0922 | 0 | 0.049507315 | 0 | 0.033011631 |
| USP26 | 0 | 0.079778176 | 0 | 0.026370729 |
| SDE2 | 0 | 0.114877236 | 0 | 0.005512139 |
| ZNF248 | 0 | 0.083903052 | 0 | 0.024784756 |

TABLE 2
Statistical analysis of MED12 G44D binding to different Mediator
modules, Related to FIG. 1C. Effect on modules was calculated
with Fisher's combined probability test on values from per
subunit two-sample t-tests with equal variances.

| | sum(−2ln(p-two | | |
|---|---|---|---|
| Module | sided)) | df | Combined two-sided p-value |
| Kinase | 35.91353872 | 8 | 1.82118E−05 |
| Middle | 2.637376289 | 8 | 0.955016839 |
| Tail | 15.10591722 | 16 | 0.516895073 |
| Head | 19.11411113 | 18 | 0.384828394 |
| MED1 or MED26 | 3.038811122 | 2 | 0.218841936 |

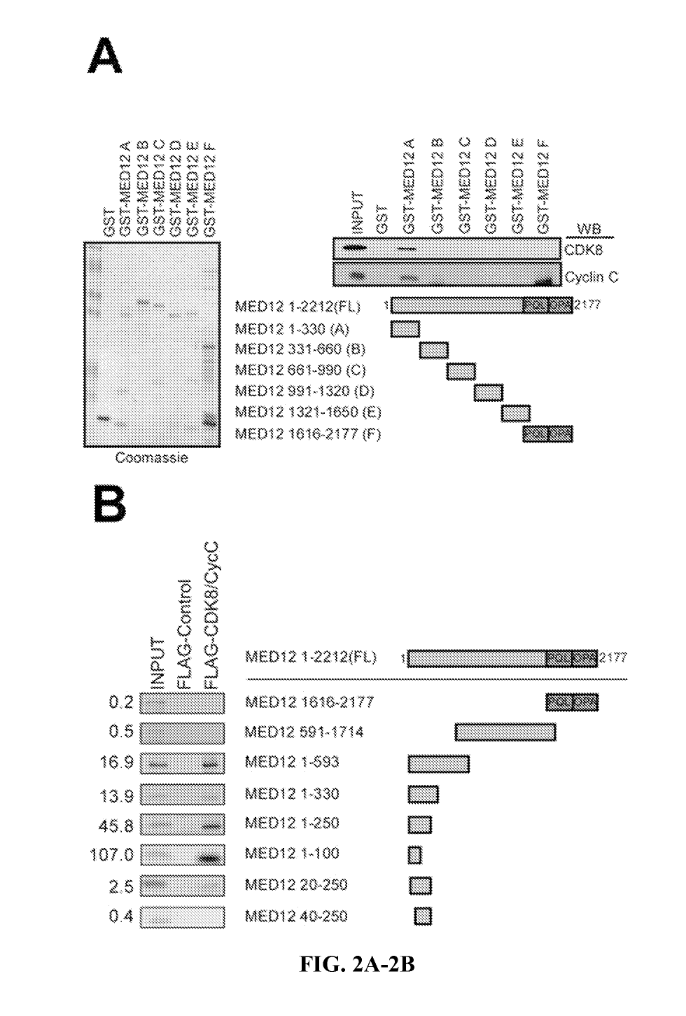

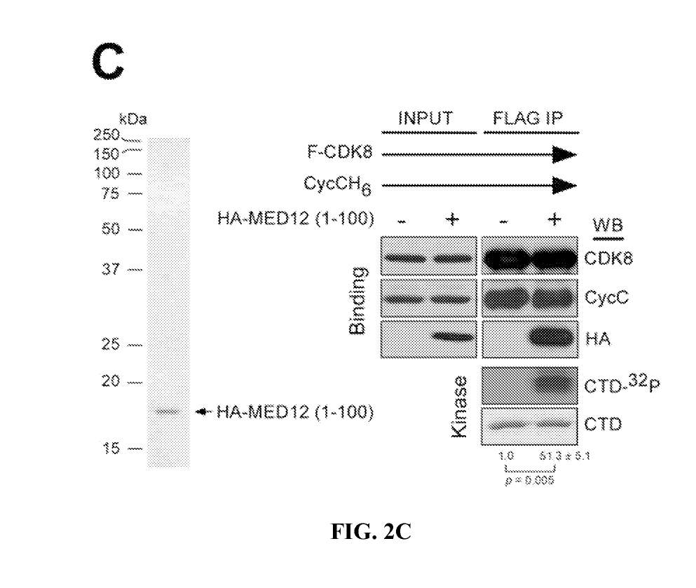

Uterine leiomyoma-linked mutations in MED12 disrupt its direct interaction with CycC-CDK8. To determine whether leiomyoma-linked mutations in MED12 disrupt its direct interaction with Cyclin C-CDK8, recombinant kinase module variants reconstituted from baculovirus expressed subunits were analyzed. CDK8 immunoprecipitates from insect cells co-expressing CDK8, Cyclin C, and either WT or mutant MED12 derivatives (L36R, Q43P, or G44S) were monitored for the presence of MED12 and the level of CDK8 kinase activity. These reconstitution assays were performed in the absence of MED13, since the latter does not appreciably impact the integrity or activity of a trimeric MED12/Cyclin C/CDK8 submodule assembly. Compared to WT MED12, all three of the MED12 leiomyoma mutants were severely compromised for both Cyclin C-CDK8 binding and activation (FIG. 1F). Cyclin C-CDK8 binding domain on MED12 was mapped to within its N-terminal 100 amino acids encoded largely by exons 1 and 2 (FIGS. 2A and 2B), and further confirmed that MED12 (1-100) binds to and activates Cyclin C-CDK8 (FIG. 2C). This suggests that exon 2 mutations in MED12 likely disrupt its Cyclin C-CDK8 binding interface as opposed to triggering conformational masking of a distant interaction site elsewhere in the protein. Together, these findings identify for the first time a common functional defect associated with uterine leiomyoma-linked mutations in MED12 and further suggest that disruption of its Cyclin C-CDK binding activity contributes to leiomyoma formation.

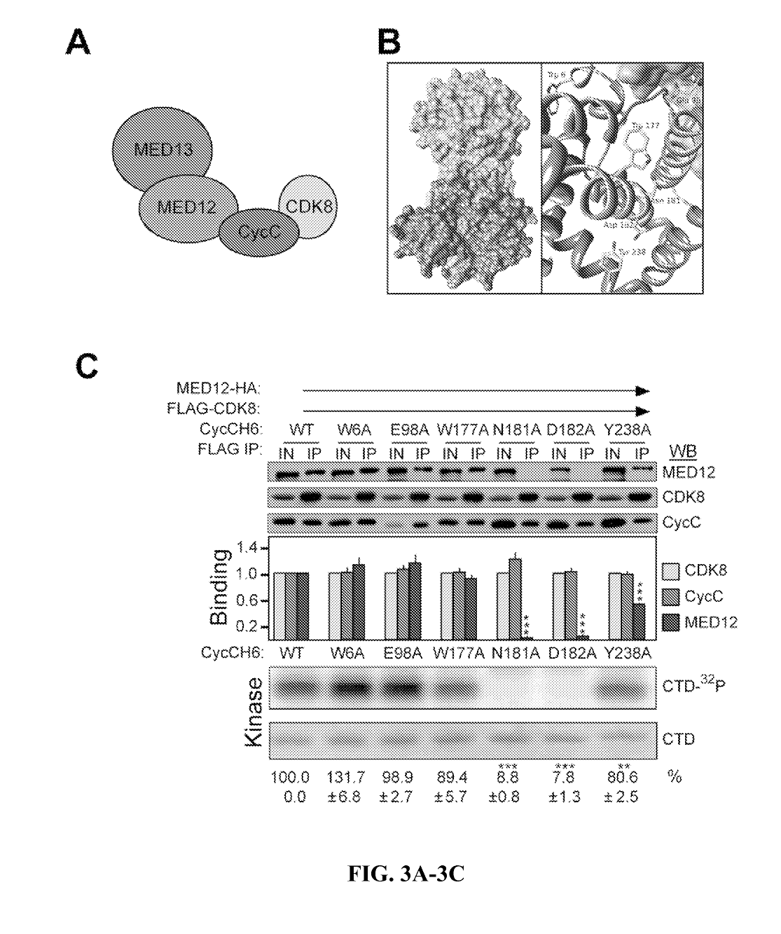

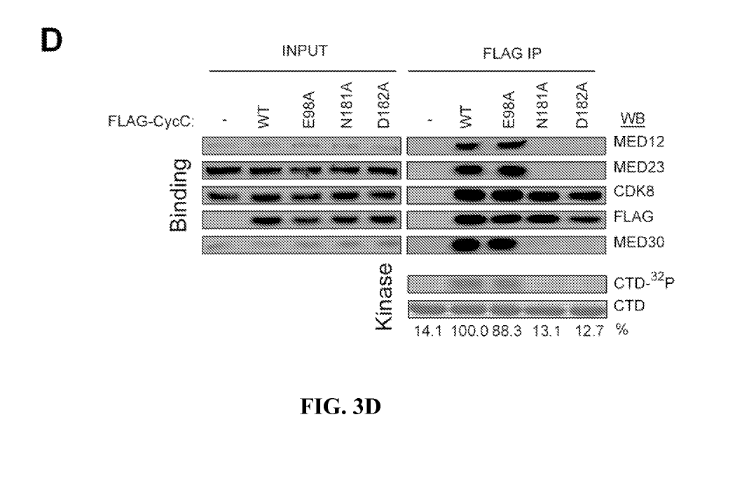

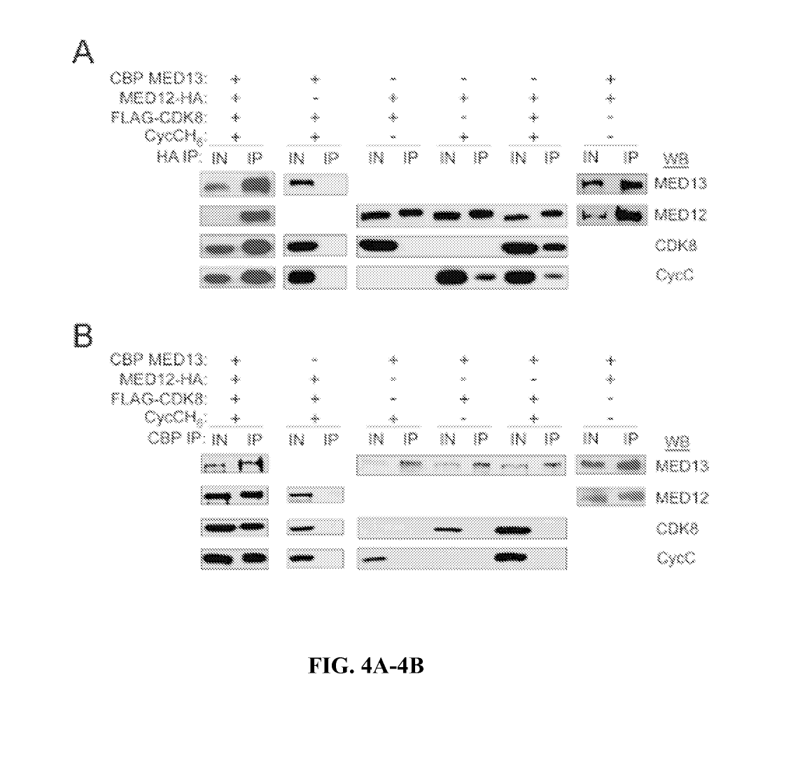

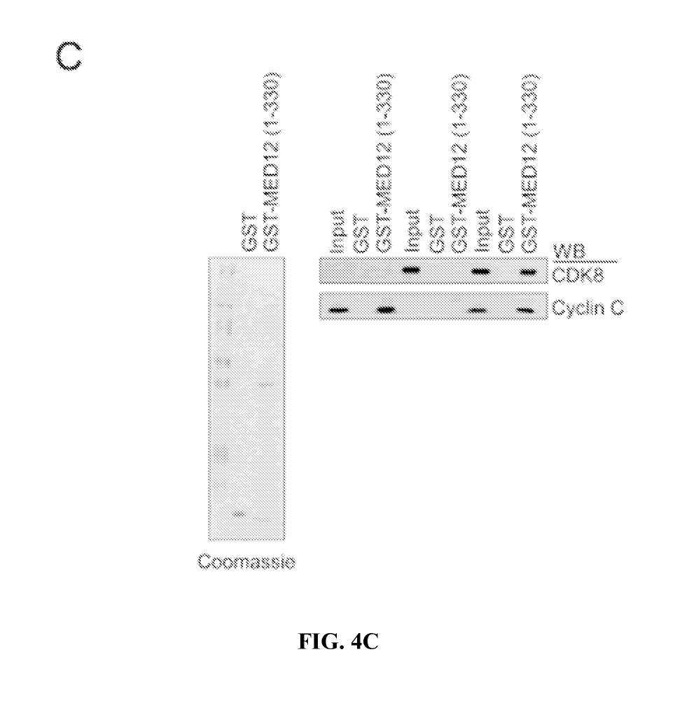

MED12 activates CDK8 through direct interaction with CycC. To clarify the molecular basis by which exon 2 mutations in MED12 disrupt its direct interaction with Cyclin C-CDK8, kinase module subunit interactions were resolved using recombinant baculovirus-expressed proteins. Immunopurification of the kinase module from insect cells expressing all possible combinations of its four constituent subunits permitted resolution of its hierarchical subunit organization. This analysis revealed that MED12 binds to Cyclin C, which in turn binds to CDK8 (FIG. 3A; FIG. 4A). MED12 also binds to MED13, which does not bind to either Cyclin C or CDK8 (FIG. 3A; FIGS. 4A and 4B). Importantly, no interaction between MED12 and CDK8 was detected in the absence of Cyclin C (FIG. 3A; FIGS. 4A and 4C), indicating that Cyclin C bridges MED12 and CDK8. These findings confirm those recently described for subunit assembly in S. cerevisiae and support a conserved molecular organization between the yeast and human kinase modules (Tsai et al., 2013, Nat. Struct. Mol. Biol., 20,611-619).

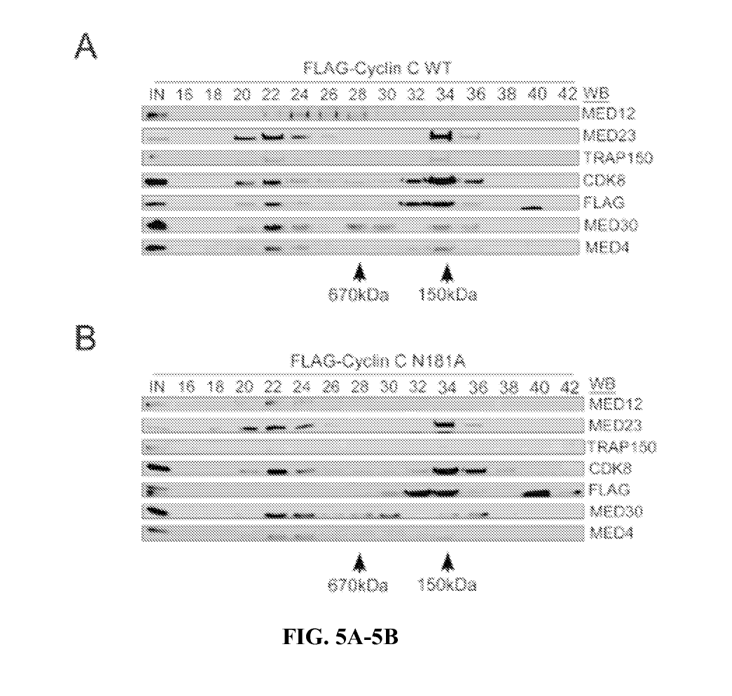

To confirm these findings in vivo, FLAG-tagged WT Cyclin C or its MED12 binding-deficient mutant derivative (N181A) was expressed in HEK293 cells and their chromatographic elution profiles by gel filtration analysis were monitored. Whereas wild-type Cyclin C coeluted along with other Mediator subunits in a ˜2 MD a Mediator peak, Cyclin C N181A was excluded separately from these fractions (FIG. 5A and 5B), indicating that surface groove mutations disrupt the association of Cyclin C with MED12, its principal anchor in Mediator. This interpretation is congruent with co-immunoprecipitation analyses from FLAG-tagged WT and mutant Cyclin C expressing cells. FLAG-specific immunoprecipitates from N181A and D182A-expressing cells harbored CDK8, but not MED12 or other Mediator subunits that were readily detected in those from WT and E98A-expressing cells (FIG. 3D). Concordantly, Cyclin C-associated CDK8 kinase activity was significantly reduced in FLAG-specific immunoprecipitates from cells expressing N181A and D182A compared to WT or E98A derivatives (FIG. 3D). Together, these results identify the Cyclin C surface groove as a principal binding interface through which MED12 both anchors Cyclin C-CDK8 into Mediator and stimulates Cyclin C-dependent CDK8 kinase activity.

Results showing that oncogenic exon 2 mutations in MED12 uncouple Cyclin C-CDK8/19 from core Mediator implicate aberrant CDK8/19 activity in uterine leiomyomagenesis and suggests new potential targets for therapeutic intervention in a tumor type that negatively impacts hundreds of millions of women worldwide.

Cloning and mutagenesis for the MED12 Flp-In™ 293 T-REx cells. Site-directed mutagenesis of MED12 to generate the G44D mutant was performed using the primers listed in Table 3. After mutagenesis the cDNA constructs were cloned into gateway compatible entry-vector and finally to pTO_HA_StrepIII_c_GW_FRT destination vector (Varjosalo et al., Cell Rep. (2013) 3:1306-20).

TABLE 3
Site-directed mutagenesis primers. Site-directed mutagenesis (Stratagene) of
MED12 to generate the L36R, Q43P, G44S, G44D mutants and of Cyclin C to
generate W6A, E98A, W177A, N181A, D182A, Y238A mutants was performed using
the following primers:

| MED12 | Forward | Reverse |
|---|---|---|
| L36R | CAGAAGGAGGATGAACGGACGGCCTT | CATTCAAGGCCGTCCGTTCATCCTCCTTCTG |
| | GAATG | |
| Q43P | GCCTTGAATGTAAAACCAGGTTTCAAT | GGCTGGTTATTGAAACCTGGTTTTACATTCAAGGC |
| | AACCAGCC | |
| G44S | GCCTTGAATGTAAAACAAAGTTTCAAT | GGCTGGTTATTGAAACTTTGTTTTACATTCAAGGC |
| | AACCAGCC | |
| G44D | CTGACGGCCTTGAATGTAAAACAAGAT | GCAGGCTGGTTATTGAAATCTTGTTTTACATTCAAGGCC |
| | TTCAATAACCAGCCTGC | GTCAG |
| CyclinC | Forward | Reverse |
| W6A | GGCAGGGAACTTTGCACAGAGCTCC | GGAGCTCTGTGCAAAGTTCCCTGCC |
| E98A | CATCCAAAGTAGCAGAATTTGGAGT | ACTCCAAATTCTGCTACTTTGGATG |
| W177A | TTCCCCTTGCAGCAAGGATAGTGAA | TTCACTATCCTTGCTGCAAGGGGAA |
| N181A | AGGATAGTGGCAGATACCTACAGA | TTCTGTAGGTATCTGCCACTATCCT |
| D182A | AGGATAGTGAATGCAACCTACAGAA | TTCTGTAGGTTGCATTCACTATCCT |
| Y238A | GTTATTTTAAAACTAGCAGAGCAGTGG | CGAAATTCTTCCACTGCTCTGCTAGTTTTAAAATAAC |
| | AAGAATTTCG | |

Affinity Purification. For each individual pulldown, a cell pellet derived from 5×15 cm dishes (approximately 5×10⁷ cells) was lysed for 10 min on ice in 5 mL HNN lysis buffer (50 mM HEPES pH 8.0, 150 mM NaCl, 5 mM EDTA, 0.5% NP-40, 50 mM NaF, 1.5 mM Na₃VO₄, 1.0 mM PMSF and 10 μL/mL protease inhibitor cocktail, Sigma). Insoluble material was removed by centrifugation at 13,000 rpm for 20 min at 4° C. 200 μL, Strep-Tactin Sepharose beads (400 μL, slurry) were transferred to a Bio-Spin chromatography column (Bio-Rad) and washed with 3×1 mL FINN buffer and 3×1 mL FINN buffer without detergent and inhibitors, and bound proteins eluted with 3×300 μL, freshly-prepared 0.5 mM D-biotin (Thermo Scientific) in FINN buffer into a fresh 1.5 ml eppendorf tube.

Mass spectrometry. Samples were prepared for LC-MS as follows: DTT was added to the eluates to a final concentration of 10 mM and the samples incubated for 1 h at 56° C. To block cysteine residues, iodoacetamide was added to a final concentration of 55 mM and the samples incubated at RT in the dark for 30 min. 1 μg trypsin was added and the samples were incubated overnight at 37° C. Tryptic digests were quenched with 10% TFA, concentrated and purified by reverse-phase chromatography MicroSpin™ Column (C18, Nest Group) and eluted with 90% CH₃CN, 0.1% TFA. The volume of the eluted sample was reduced to approximately 2 μL in a vacuum centrifuge and reconstituted to a final volume of 40 μl with 0.1% TFA, 1% CH₃CN and vortexed thoroughly.

Mass spectrometry analysis was performed on an Orbitrap Elite ETD mass spectrometer (Thermo Scientific, Waltham, Mass.) using the Xcalibur version 2.7.1 coupled to an Thermo Scientific nLCII nanoflow system (ThermoScientific) via a nanoelectrospray ion source. Solvents for LCMS separation of the digested samples were as follows: solvent A consisted of 0.1% formic acid in water (98%) and acetonitrile (2%) and solvent B consisted of 0.1% formic acid in acetonitrile (98%) and water (2%). From a thermostatted microautosampler, 8 μl of the tryptic peptide mixture (corresponding to 20% of the final SH-TAP eluate) were automatically loaded onto a 15 cm fused silica analytical column with an inner diameter of 75 μm packed with C18 reversed phase material (Thermo Scientific) and the peptides were eluted from the analytical column with a 40 minute gradient ranging from 5 to 35% solvent B, followed by a 10 minute gradient from 35 to 80% solvent B at a constant flow rate of 300 nl/min. The analyses were performed in a data dependent acquisition mode using a top 10 collision-induced dissociation (CID) method. Dynamic exclusion for selected ions was 30 seconds. No lock masses were employed. Maximal ion accumulation time allowed on the Orbitrap Elite ETD in CID mode was 100 ms for MSn in the Ion Trap and 200 ms in the FTMS. Automatic gain control was used to prevent overfilling of the ion traps and were set to 10,000 (CID) in MSn mode for the Ion Trap, and 106 ions for a full FTMS scan. Intact peptides were detected in the Orbitrap at 60,000 resolution. Peak extraction and subsequent protein identification was achieved using Proteome Discoverer software (Thermo Scientific, Waltham, Mass.). Calibrated peak files were searched against the human component of UniProtKB/SwissProt database (available on the worldwideweb at uniprot.org) by a SEQUEST search engine. Error tolerances on the precursor and fragment ions were ±15 ppm and ±0.6 Da, respectively. Database searches were limited to fully-tryptic peptides with maximum 1 missed cleavage, carbamidomethyl cysteine and methionine oxidation were set as fixed and variable modifications, respectively. The normalization of protein abundance is described in Table 1.

Kinase assays. For in vitro kinase assays, insect cell lysates expressing MED12-HA (WT or mutants), CDK8-FLAG, and CycCH6 (WT or mutants) were combined in different combinations and subjected to FLAG IP for 1 hour, at 4° C. in 200 mM NaCl D Buffer. Immune complexes were washed in 200mM NaCl D buffer and subjected to a kinase assay containing 25 mM Tris pH7.5, 20 mM MgCl₂, 2.5 mCi [γ-³²P]-ATP and 2 μg of purified GST-3X-CTD. Reactions were incubated for 30 minutes at 30° C., eluted in Laemmli sample buffer, processed by SDS-12% PAGE, stained with Coomassie blue and visualized by phosphorimager analysis. ³²P-labeled GST-3XCTD was quantified using ImageQuant software.

For in vivo derived kinase assays, HEK293 cells were transfected with pCDNA3.1-3×FLAG Cyclin C (WT or mutant) or 3×FLAG MED12 (WT or mutant) plasmids and nuclear extracts were harvested 48 hours later. Extracts were subjected to FLAG IP in 200 mM D buffer overnight at 4° C. Immune complexes were washed and subjected to a kinase assay containing 25 mM Tris pH7.5, 20 mM MgCl2, 2.5 mCi [γ-³²P]-ATP and 2 μg of purified GST-3X-CTD. Reactions were incubated for 30 minutes at 30° C., eluted in Laemmli sample buffer, processed by SDS-12% PAGE, stained with Coomassie stain and visualized by phosphorimager analysis. ³²P-labeled GST-3X-CTD levels were quantified using ImageQuant software.

1. A method of treatment comprising administering a polypeptide having an amino acid sequence at least 90% identical to the amino acid sequence of MAAFGILSYEHRPLKRPRLGPPDVYPQDPKQKEDELTALNVKQGFNNQPAVSGDEH GSAK NVSFNPAKISSNFSSIIAEKLRCNTLPDT (SEQ ID NO:1) to a subject having or suspected of having uterine leiomyomas.

2. The method of claim 1, wherein the polypeptide is administered by expression from an expression cassette.

3. The method of claim 2, wherein the expression cassette is comprised in a viral expression vector.

4. A peptide comprising an amino acid sequence at least 90% identical to the amino acid sequence of MAAFGILSYEHRPLKRPRLGPPDVYPQDPKQKEDELTALNVKQGFNNQPAVSGDEH GSAK NVSFNPAKISSNFSSIIAEKLRCNTLPDT (SEQ ID NO:1).

5. The peptide of claim 1, wherein the peptide has the amino acid sequence of SEQ ID NO:1.

6. A pharmaceutical composition comprising the peptide of claim 1.

7. A method treating uterine leiomyomas comprising administering a peptide of claim 1 to a subject having or suspected of having uterine leiomyomas.


SEQUENCE LISTING (21 sequences)

SEQ ID NO: 1 - protein, 89 aa, Homo sapiens
MAAFGILSYEHRPLKRPRLGPPDVYPQDPKQKEDELTALNVKQGFNNQPAVSGDEHGSAKNVSFNPAKISSNFSSIIAEKLRCNTLPDT

SEQ ID NO: 2 - DNA, 31 bp, Artificial Sequence
  Synthetic oligonucleotide
cagaaggaggatgaacggacggccttgaatg

SEQ ID NO: 3 - DNA, 31 bp, Artificial Sequence
  synthetic oligonucleotide
cattcaaggccgtccgttcatcctccttctg

SEQ ID NO: 4 - DNA, 35 bp, Artificial Sequence
  synthetic oligonucleotide
gccttgaatgtaaaaccaggtttcaataaccagcc

SEQ ID NO: 5 - DNA, 35 bp, Artificial Sequence
  synthetic oligonucleotide
ggctggttattgaaacctggttttacattcaaggc

SEQ ID NO: 6 - DNA, 35 bp, Artificial Sequence
  synthetic oligonucleotide
gccttgaatgtaaaacaaagtttcaataaccagcc

SEQ ID NO: 7 - DNA, 35 bp, Artificial Sequence
  synthetic oligonucleotide
ggctggttattgaaactttgttttacattcaaggc

SEQ ID NO: 8 - DNA, 44 bp, Artificial Sequence
  synthetic oligonucleotide
ctgacggccttgaatgtaaaacaagatttcaataaccagcctgc

SEQ ID NO: 9 - DNA, 44 bp, Artificial Sequence
  synthetic oligonucleotide
gcaggctggttattgaaatcttgttttacattcaaggccgtcag

SEQ ID NO: 10 - DNA, 25 bp, Artificial Sequence
  synthetic oligonucleotide
ggcagggaactttgcacagagctcc

SEQ ID NO: 11 - DNA, 25 bp, Artificial Sequence
  synthetic oligonucleotide
ggagctctgtgcaaagttccctgcc

SEQ ID NO: 12 - DNA, 25 bp, Artificial Sequence
  synthetic oligonucleotide
catccaaagtagcagaatttggagt

SEQ ID NO: 13 - DNA, 25 bp, Artificial Sequence
  synthetic oligonucleotide
actccaaattctgctactttggatg

SEQ ID NO: 14 - DNA, 25 bp, Artificial Sequence
  synthetic oligonucleotide
ttccccttgcagcaaggatagtgaa

SEQ ID NO: 15 - DNA, 25 bp, Artificial Sequence
  synthetic oligonucleotide
ttcactatccttgctgcaaggggaa

SEQ ID NO: 16 - DNA, 24 bp, Artificial Sequence
  synthetic oligonucleotide
aggatagtggcagatacctacaga

SEQ ID NO: 17 - DNA, 25 bp, Artificial Sequence
  synthetic oligonucleotide
ttctgtaggtatctgccactatcct

SEQ ID NO: 18 - DNA, 25 bp, Artificial Sequence
  synthetic oligonucleotide
aggatagtgaatgcaacctacagaa

SEQ ID NO: 19 - DNA, 25 bp, Artificial Sequence
  synthetic oligonucleotide
ttctgtaggttgcattcactatcct

SEQ ID NO: 20 - DNA, 37 bp, Artificial Sequence
  synthetic oligonucleotide
gttattttaaaactagcagagcagtggaagaatttcg

SEQ ID NO: 21 - DNA, 37 bp, Artificial Sequence
  synthetic oligonucleotide
cgaaattcttccactgctctgctagttttaaaataac